(19) United States
(12) Patent Application Publication
(10) Pub. No.: US 2013/0230859 A1
(43) Pub. Date: Sep. 5, 2013

(54) USE OF BLOOD GROUP STATUS

(71) Applicants: Pirjo Wacklin, Helsinki (FI); Jaana Mättö, Helsinki (FI); Harri Mäkivuokko, Kirkkonummi (FI); Jukka Partanen, Helsinki (FI)

(72) Inventors: Pirjo Wacklin, Helsinki (FI); Jaana Mättö, Helsinki (FI); Harri Mäkivuokko, Kirkkonummi (FI); Jukka Partanen, Helsinki (FI)

(73) Assignee: SUOMEN PUNAINEN RISTI VERIPALVELU, Helsinki (FI)

(21) Appl. No.: 13/812,580

(22) PCT Filed: Jul. 26, 2011
(86) PCT No.: PCT/FI2011/050675

§ 371 Date: Jan. 28, 2013

(30) Foreign Application Priority Data
Jul. 26, 2010 (FI) 20105824

(51) Int. Cl. C12Q 1/68

(52) U.S. Cl. CPC C12Q 1/689
USPC 435/6.12; 435/252.9

(57) ABSTRACT

The present invention relates to use of the non-secretor/secretor blood group status of an individual as a criterion for need of Lactobacillus/LAB-enriched probiotic supplementation. The present invention relates also to method of assessing the need of an individual for Lactobacillus/LAB-enriched probiotic supplementation by determining the secretory blood group status of the individual.

### FIELD OF THE INVENTION

The present invention relates to use of the non-secretor/secretor blood group status of an individual as a criterion for need of Lactobacillus/lactic acid bacteria (LAB)-enriched probiotic supplementation. The present invention relates also to a method of assessing the need of an individual for Lactobacillus-/LAB enriched probiotic supplementation by determining the secretor blood group status of the individual. The present invention relates further to microbial compositions. Also, the invention relates to the use of prebiotics, molecular compounds or additional supportive bacteria strains, to increase the number of, and/or to augment the growth and/or functionality of probiotics containing Lactobacillus/LAB in an individual with non-secretor blood group.

### BACKGROUND OF THE INVENTION

Human intestinal tract is colonised with over 500 bacterial species, whose total number can exceed trillions of microbial cells in the colon. This microbiota in the large intestine is mainly composed of Firmicutes and Bacteroides phyla, which make up over 75% and 16% of total microbes in the gut (Eckburg P B, et al. Science 308:1635-8, 2005; Tap J, et al. Environ Microbiol 11:2574-84, 2009). Within Firmicutes phyla, Clostridium and its close relatives dominate. Bacteroides species found in the gut mainly belong to B. fragilis group. In spite of low diversity at the microbial phyla level, the gut microbiota composition among individuals is highly variable at species and strain level. The core microbiota consisted mainly species of Bacteroides and clostridia; in addition, one Bifidobacterium spp and one Coprobacillus spp. were included in the core.

The microbiota has an important role in human health. It contributes to the maturation of the gut tissue, to host nutrition, pathogen resistance, epithelial cell proliferation, host energy metabolism and immune response (e.g. Dethlefsen et al. Trends Ecol Evol 21(9):517-23, 2006; Round and Mazmanian, Nat Rev Immunol 9(5):313-23, 2009; Round and Mazmanian, Proc Natl Acad Sci USA doi/10.1073/pnas.0909122107, 2010). An altered composition and diversity of gut microbiota have been associated to several diseases (Round and Mazmanian, 2009), such as inflammatory bowel disease, IBD (Sokol et al. Proc Natl Acad Sci USA 105(43):16731-6, 2008.), irritable bowel syndrome (Mättö et al. FEMS Immunol Med Microbiol 43(2):213-22, 2005.), rheumatoid arthritis (Vaahtovuo et al. J Rheumatol 35(8):1500-5, 2008), atopic eczema (Kalliomaki M and Isolauri E., Curr Opin Allergy Clin Immunol 3(1):15-2, 2003), and type 1 diabetes (Wen et al. Nature 455(7216):1109-13, 2008). Little is known, however, which species mediate beneficial responses. A decrease in the number of Faecalibacterium prausnitzii has been observed in IBD and evidence indicates that F. prausnitzii has anti-inflammatory effects in vitro and in vivo (Sokol et al. 2008).

The role of host genes on composition of gut microbes has been supported by twin studies, which showed that monozygotic twins have more similar gut microbiota than dizygotic twins or unrelated persons (Zoetendal et al. Microbial Ecology in Health and Disease 13(3):129-3, 2001). However, little is known which genes determine or regulate the microbial composition. Some gut microbes e.g. Helicobacter pylori and pathogenic species of bacteria and viruses have been shown to use ABO blood group antigens as adhesion reseptors (Boren et al. Science 1993, 262, 1892-1895). Some microbes e.g. Bifidobacteria and Bacteroides thetaiotaomicron are also able to utilize blood group antigens or glycans found in ABO and Lewis antigens.

The ABO blood group antigens are not present in the mucus of all individuals. These individuals, said to have the ‘non-secretor’ blood group, do not have the functional FUT2 gene needed in the synthesis of secreted blood group antigens (Henry et al. Vox Sang 1995; 69(3):166-82). Hence, they do not have ABO antigens in secretions or mucosa. Those with blood group ‘secretor’ have the antigens on mucosa. In most populations, the frequency of non-secretor individuals is substantially lower than that of secretor status; about 15-26% of Scandinavians are non-secretors (Eriksson et al. Ann Hum Biol. 1986 May-June; 13(3):273-85). The secretor/non-secretor status can be regarded as a normal blood group system and the phenotype can be determined using standard blood banking protocols (Henry et al. Vox Sang 1995; 69(3):166-82). The genotype, that is, the major mutation in the FUT2 gene causing the non-secretor phenotype in the European populations (Silva et al. Glycoconj 2010; 27:61-8) has been identified. Non-secretor phenotype has been demonstrated to be genetically associated for example, with an increased risk for Crohn's disease (McGovern et al. Hum Molec Genet 2010 Advance Access Published Jun. 22, 2010), with high vitamin B12 levels in the blood (Tanaka et al Am J Hum Genet 2009; 84:477-482), with resistance to Norovirus infection (Thorven et al J Virol 2005; 79: 15351-15355), with susceptibility to HI virus infection (Ali et al 2000, J Infect Dis 181: 737-739), with experimental vaginal candidiasis (Hurd and Domino Infection Immunit 2004; 72: 4279-4281), with an increased risk for asthma (Ronchetti et al. Eur Respir J 2001; 17: 1236-1238), with urinary tract infections (Sheinfeld et al N Engl J Med 1989; 320: 773-777), and with an animal hemorrhagic disease virus (Guillon et al. Glycobiology 2009; 19: 21-28).

The beneficial effects of certain microbial species/strains on maintaining or even improving the balance of the gut and growing evidence of their health effects on intestinal inflammatory diseases have lead to great interest on modulation of gut microbiota. Gut microbiota can be modulated by probiotics, which currently belong mainly to Bifidobacteria and Lactobacillus genera. However, the efficacy of probiotics seems to vary—for reasons not understood (e.g. Bezkorovainy, Am J Clin Nutr 73(suppl): 399s-404s, 2001).

Many probiotic supplements and products currently on the market are ineffective in promoting the desired health effects among most individuals. Thus, there is a continuous need for microbial and/or probiotic products that are able to mediate the health effects of the microbes more efficiently.

### BRIEF DESCRIPTION OF THE INVENTION

The invention is based on the finding that blood group secretor/non-secretor status of an individual was surprisingly shown to determine the diversity and repertoire of Lactobacillus/LAB in the gut of the individual. As Lactobacillus is a major microbe genus in probiotics, the present invention can be used to improve the efficacy of probiotics and to identify individuals with a need for an increased dosage or diversity of Lactobacillus/LAB in probiotics, and/or to identify individuals with a need for supplementation of prebiotics supporting Lactobacillus/LAB. Hence, the current invention provides a novel and effective means for optimizing the bacterial, especially Lactobacillus/LAB content of a microbial or probiotic composition.

An object of the invention is use of secretor/non-secretor blood group status of an individual in assessing the need for Lactobacillus/LAB-enriched probiotic supplementation, i.e., as a criterion for Lactobacillus/LAB-enriched probiotic supplementation. The present invention relates also to method of assessing the need of an individual for Lactobacillus/LAB-enriched probiotic supplementation by determining the secretor/non-secretor status of the individual. Also, an object of the invention is the use of secretor/non-secretor blood group status of an individual in assessing the need for supplementation of Lactobacillus/LAB-supporting prebiotics, molecular compounds or additional supportive bacteria strains. A further object of the present invention is a method of assessing the need of an individual for supplementation of Lactobacillus/LAB-supporting prebiotics, molecular compounds or additional supportive bacteria strains by determining the secretory status of the individual. Such Lactobacillus/LAB-supporting prebiotics, molecular compounds or additional supportive bacteria strains contribute to increase the number of, and/or to augment the growth and/or functionality of Lactobacillus/LAB in individuals with non-secretor status.

A further object of the present invention is a use of the secretor/non-secretor blood group status of an individual in estimating a dose of Lactobacillus/LAB supplementation needed for a desired effect. Another further object of the present invention is a use of the secretor/non-secretor blood group status of an individual in estimating a dose of supplementation of Lactobacillus/LAB supporting prebiotics, molecular compounds or additional supportive bacteria strains needed for a desired effect. The present invention also relates to a method of estimating a dose of Lactobacillus/LAB supplementation needed for a desired effect by determining the secretor/non-secretor status of the individual. The present invention further relates to a method of estimating a dose of supplementation of Lactobacillus/LAB supporting prebiotics, molecular compounds or additional supportive bacteria strains needed for a desired effect by determining the secretor/non-secretor status of the individual.

Additionally, the present invention relates to use of the secretor/non-secretor status of an individual to augment the stabilisation of mucosal microbiota of an individual, in particular its Lactobacillus/LAB composition, in disorders related to, or after treatments leading to unbalance of mucosal microbiota, such as celiac disease and graft-versus-host disease.

Further objects of the present invention are methods of tailoring a microbial or probiotic composition based on the spectrum of Lactobacillus/LAB found from the intestine of at least one individual with non-secretor blood group phenotype or with secretor blood group phenotype.

An object of the invention is also a microbial and/or probiotic composition tailored based on the spectrum of Lactobacillus/LAB found from the intestine of at least one individual with non-secretor blood group phenotype or with secretor blood group phenotype. Further, an object of the present invention is the microbial composition for use in the treatment and/or prevention of disorders related to, or after treatments leading to unbalance of mucosal microbiota.

The objects of the invention are achieved by the compositions, methods and the uses set forth in the independent claims. Preferred embodiments of the invention are described in the dependent claims.

Other objects, details and advantages of the present invention will become apparent from the following drawings, detailed description and examples.

### DESCRIPTION OF THE DRAWING

FIG. 1 shows the richness, that is, the number of DGGE bands or genotypes detected in Lactobacillus-DGGE and the Simpson diversity index in the samples studied. The non-secretors had a lower number of Lactobacillus/LAB genotypes than secretors and a lower Simpson diversity index; the significance in the difference between non-secretor (NSS, n=6) and secretor (SS, n=49) samples was evaluated by ANOVA.

### DETAILED DESCRIPTION OF THE INVENTION

The present invention is based on the finding that the intestinal Lactobacillus/lactic acid bacteria (LAB) populations differ between blood group secretor and non-secretor individuals. Individuals with non-secretor blood group have a reduced diversity of Lactobacillus/LAB in their intestinal bacterial population. Moreover, non-secretor individuals have lower proportion of acid and/or bile tolerant Lactobacillus/LAB in their intestine. However, several LAB genotypes representing several Lactobacillus spp. species, pediococci and Weissella spp. occur more frequently in non-secretor individuals than in secretor individuals. Further, Lactobacillus genotypes having DGGE band positions 6.30% and 26.80% were more commonly detected in non-secretor individuals than secretor individuals. These findings can be used as a basis for targeted modulation of the Lactobacillus/LAB populations in the non-secretor/secteror individuals and as a criterion for Lactobacillus/LAB enriched probiotic supplementation.

The term ‘probiotic’ here refers to any bacterial species, strain or their combinations, with health supportive effects, not limited to currently accepted strains or to intestinal effects. The term ‘prebiotic’ here refers to any compound, nutrient, or additional microbe applied as a single additive or as a mixture, together with probiotics or without probiotics, in order to augment a desired probiotic health effect or to stimulate the growth and activity of those bacteria which are assumed to be beneficial to the health of the host body. LAB are defined as the genera that comprise Lactobacillus, Leuconostoc, Pediococcus, Lactococcus, and Streptococcus as well as the more peripheral Aerococcus, Carnobacterium, Enterococcus, Oenococcus, Sporolactobacillus, Tetragenococcus, Vagococcus, and Weisella; these belong to the order Lactobacillales.

The present invention relates to a use of the secretor/non-secretor blood group status of an individual in assessing the need for Lactobacillus/LAB supplementation, especially Lactobacillus/LAB enriched probiotic supplementation. As individuals with non-secretor blood group were found to have a lower diversity of Lactobacillus/LAB, they need higher dosages of Lactobacillus/LAB containing probiotics to achieve levels similar as those of secretors. As the frequency of selected Lactobacillus/LAB genotypes differ between secretor and non-secretor individuals the probiotic supplementation needs to be tailored at genotype and/or strain level. In one embodiment, the Lactobacillus/LAB supplementation comprises Lactobacillus genotypes having DGGE band positions 6.30% and/or 26.80%. In another embodiment, the Lactobacillus/LAB supplementation comprises L. plantarum and/or L. acidophilus. In a further embodiment, the Lactobacillus/LAB supplementation comprises L. plantarum having DGGE band position 6.30% and/or L. acidophilus having DGGE band position 26.80%.

The present invention also relates to a use of the secretor/non secretor blood group status of an individual in assessing the need for Lactobacillus/LAB-supporting prebiotic supplementation. Thus, in one embodiment of the invention, a prebiotic supporting the growth or effects of Lactobacillus/LAB is added in increased levels into a probiotic composition.

The present invention also relates to a method of assessing the need of an individual for Lactobacillus/LAB supplementation, especially Lactobacillus/LAB-enriched probiotic supplementation, by determining the secretor/non-secretor status of the individual. Further, the present invention relates to a method of assessing the need of an individual for supplementation of Lactobacillus/LAB-supporting prebiotics by determining the secretor/non secretor blood group status of the individual. In one embodiment, the Lactobacillus/LAB supplementation comprises Lactobacillus genotypes having DGGE band positions 6.30% and/or 26.80%. In another embodiment, the Lactobacillus/LAB supplementation comprises L. plantarum and/or L. acidophilus. In a further embodiment, the Lactobacillus/LAB supplementation comprises L. plantarum having DGGE band position 6.30% and/or L. acidophilus having DGGE band position 26.80%. In addition, in one embodiment the Lactobacillus/LAB supplementation comprises L. plantarum having DNA sequence encoding the 16S rRNA identified as SEQ ID NO:1 and/or L. acidophilus having DNA sequence encoding the 16S rRNA identified as SEQ ID NO:2. In one embodiment, the microbial or probiotic supplementation comprises L. plantarum having DGGE band position 6.30% and DNA sequence encoding the 16S rRNA identified as SEQ ID NO:1. In another embodiment, the microbial or probiotic supplementation comprises L. acidophilus having DGGE band position 26.80% and DNA sequence encoding the 16S rRNA identified as SEQ ID NO:2. In a further embodiment, the microbial supplementation comprises L. plantarum having DGGE band position 6.30% and DNA sequence encoding the 16S rRNA identified as SEQ ID NO:1 together with L. acidophilus having DGGE band position 26.80% and DNA sequence encoding the 16S rRNA identified as SEQ ID NO:2.

The present invention further relates to uses of the secretor/non-secretor status of an individual in estimating a dose of Lactobacillus/LAB supplementation needed for a desired effect or that of Lactobacillus/LAB-supporting prebiotic needed for a desired probiotic effect. Typically individuals of non-secretor phenotype should need higher doses and different genotypes of probiotics than those with the secretor phenotype. In addition, individuals of non-secretor phenotype should need higher doses of Lactobacillus/LAB-supporting prebiotics than those with the secretor phenotype.

The secretor/non-secretor status can be determined in vitro, for example, from a sample of saliva, using standard blood grouping methods or from the genomic DNA sample taken from an individual by determining adequate mutations in the FUT2 gene (Silva et al. Glycoconjugate Journal 2009, DOI 10.1007/s10719-009-9255-8). The major mutation in European populations seems to be W143X, but other mutations have also been described and obviously, more can be identified when additional samples are analysed.

In one embodiment of the present invention, the secretor/non-secretor status can be used to augment the stabilisation of mucosal microbiota composition, in particular its Lactobacillus/LAB composition, of an individual after disorders or treatments known to disturb the balance of mucosal microbiota. Examples of these comprise treatments with broad spectrum antibiotics, irradiation or cytotoxic therapies related to cancer treatments or bone marrow transplantation and/or gastroenterological infections by e.g. Noro-virus or Helicobacter. The present invention is further targeted to treatment of diseases or traits, having the FUT2 gene (i.e. the secretor blood group status) as a genetic susceptibility factor. These comprise, just to give examples, low levels of vitamin B12 in the blood, various clinical forms of inflammatory bowel disease, urinary tract infections, vaginal candidiasis, Noro- and HI-virus infections and infections by hemorrhagic viruses. It is likely that a higher number of diseases will be identified in the future by screening the FUT2 locus. In such cases, probiotic treatments can used to direct or change the microbiological balance in the mucous tissue, such as in the gut, toward more Lactobacillus/LAB-rich. Individuals with the non-secretor phenotype typically require higher dosages and/or preparations with more diverse Lactobacillus/LAB strains than secretors. Thus, the present invention relates also to use of the secretor/non-secretor status of an individual to augment the stabilisation of mucosal microbiota, in particular its Lactobacillus/LAB composition in disorders or conditions related to, or after treatments leading to unbalance of mucosal microbiota. The Lactobacillus/LAB composition can also be used to prevent the microbiota imbalance. In one embodiment, the Lactobacillus/LAB composition comprises Lactobacillus genotypes having DGGE band positions 6.30% and/or 26.80%. In another embodiment, the Lactobacillus/LAB composition comprises L. plantarum and/or L. acidophilus. In a further embodiment, the Lactobacillus/LAB composition comprises L. plantarum having DGGE band position 6.30% and/or L. acidophilus having DGGE band position 26.80%. In addition, in one embodiment the Lactobacillus/LAB composition comprises L. plantarum having DNA sequence encoding the 16S rRNA identified as SEQ ID NO:1 and/or L. acidophilus having DNA sequence encoding the 16S rRNA identified as SEQ ID NO:2.

The present invention can be targeted to stabilisation of the gut microbiota of an individual using Lactobacillus/LAB bacteria that were found to be typical to individuals with the same secretor/non-secretor phenotype as the individual to be treated and/or a bacterial product enriched with those Lactobacillus/LAB bacteria that were found to be typical to individuals with the same secretor/non-secretor phenotype as the individual to be treated. The stabilisation can be either prophylactic, i.e. started before treatments disturbing the balance of gut microbiota, or it can be started once the symptoms develop. Further, the present invention can be targeted to increasing the number of those beneficial Lactobacillus/LAB bacteria scarcely found in individuals with the same secretor/non-secretor phenotype as the individual to be treated by adding the said bacteria into a product.

In one embodiment, the invention is related to a microbial composition, especially Lactobacillus/LAB probiotic composition, for use in the treatment and/or prevention of disorders related to, or after treatments leading to unbalance or disturbed balance of mucosal microbiota, such as stem cell transplantation and/or subsequent graft-versus-host disease.

In another embodiment, the invention is related to Lactobacillus/LAB probiotic composition for prevention or treatment of inflammatory bowel disease (IBD). IBD is an excellent target disease for the invention as an altered microbiota composition in the patients has been reported (Sokol et al. Inflamm Bowel Dis. 2006 February; 12(2):106-11). Furthermore, it is established (McGovern et al. Hum Molec Genet 2010; 19(17): 3468-76) that the non-secretor phenotype, i.e. FUT2 gene defect, confers genetic susceptibility to IBD. Hence, it is plausible that the composition according to the present invention is particularly effective in IBD. The treatment can be targeted to a relief of the symptoms and/or to prevention of relapses and/or to increasing the overall quality of life in IBD. It also may be administered together with other currently known drugs for IBD. The composition in one embodiment is targeted to those IBD patients with the non-secretor phenotype.

In another embodiment, the invention is related to Lactobacillus/LAB composition for prevention and treatment of microbial infections i.e. diarrhoea and respiratory tract infections as also in these indications therapeutic potential of probiotics (Chouraqui et al. J Pediatr Gastroenterol Nutr. 2004 March; 38(3):242-3; de Vrese et al. Clin Nutr. 2005 August; 24(4):479-80), and an increased frequency in non-secretor individuals (Ahmed et al. 2009 Infect Immun. 2009 77(5):2059-64; Raza et al. BMJ. 1991, 303(6806):815-8) have been described.

In another embodiment, the invention is related to Lactobacillus/LAB probiotic composition for prevention and treatment of irritable bowel syndrome as disturbed microbiota (Mättö et al. FEMS Immunol Med Microbiol. 2005 43(2):213-22) and potential of probiotic products have been described in IBS (Kajander et al. Aliment Pharmacol Ther. 2008 27(1):48-57).

In yet another embodiment, the invention is related to Lactobacillus/LAB probiotic composition for prevention and treatment of allergy/atopy in children. It is established that babies who develop allergy have disturbed microbiota in their intestine during the first year of life (Björksten et al. J Allergy Clin Immunol. 2001 108(4):516-20). Moreover, it has been shown bacterial composition in the milk of allergic mothers differs from that of non-allergic mothers (Grönlund et al. Clin Exp Allergy. 2007, 37(12):1764-72). Probiotic products have shown potential in prevention of atopic eczema (Yoo et al. Proc Am Thorac Soc (2007) 4, 277-282).

In one embodiment of the invention, the Lactobacillus/LAB probiotic composition or the supplement comprising the composition is particularly suitable and effective, in use for the enhancement of the diversity and numbers of intestinal bacteria, or balancing the microbiota in an individual suffering from celiac disease. It has recently been demonstrated that patients with celiac disease and its different clinical forms have disturbed gut microbiota (Wacklin P, Pusa E, Kaukinen K, Mäki M, Partanen J, and Mättö J. Composition of the mucosa-associated microbiota in the small intestine of coeliac disease patients and controls. A poster presented in the Rowett-INRA Gut Microbiology-conference, Aberdeen, UK, Jun. 6, 2010). Briefly, to evaluate differences between disease symptom groups, intestinal microbiota compositions were assessed from mucosal biopsy samples from 26 coeliac disease patients (further sub-grouped to gastrointestinal, anemia and dermatitis herpetiformis symptom groups) and 25 healthy controls. Intestinal microbiota of the coeliac disease patients representing different symptom groups clustered in clearly distinct groups. The finding indicates that the microbiota composition is variable between individuals and in intestinal disorders disease-group related differences in the microbiota composition exist, which should be taken into consideration in applications targeting for balancing or modulating the intestinal microbiota of individuals suffering from immunological gastrointestinal disorders.

In another embodiment of the invention, the secretor/non-secretor status can be used to augment the stabilisation of mucosal microbiota composition, especially the Lactobacillus/LAB composition of an individual after disorders or treatments known to disturb the balance of mucosal microbiota. Examples of these comprise treatments with broad spectrum antibiotics, irradiation or cytotoxic therapies related to cancer treatments or bone marrow transplantation or its complications such as graft-versus-host disease and/or gastroenterological infections by e.g. Noro-virus or Helicobacter.

It has recently been demonstrated that the balance of the gut microbiota in patients who have received bone marrow transplantantation is disturbed (Pusa E, Taskinen M, Lähteenmäki K, Kaartinen T, Partanen J, Vettenranta K, Mättö J. Do intestinal bacteria or donor derived responsiveness to microbial stimuli play a role post allogeneic HSCT? A poster presented in the 7^{th} Meeting of the EBMT Paediatric Diseases Working Party, 2-4 June, 2010 Helsinki, Finland). To evaluate the intestinal microbiota disturbance following chemotherapy or irradiation treatments related to treatments of malignant diseases intestinal microbiota composition of hematopoietic transplantation patients was monitored. Faecal samples were collected from pediatric HSCT patients both before transplantation and at different time points, up to 6 months, after the transplantation and their donors. PCR-DGGE analysis revealed remarkable instability of the intestinal microbiota after transplantation. The similarity of the dominant microbiota was extremely low during the first month after transplantation while up to 94% similarity was detected between the samples obtained 4-6 months from the transplantation. PCR-DGGE specific bacterial group targeted primers revealed absence of several common intestinal bacteria in several samples obtained within one month from transplantation. These findings indicate a drastic disturbance of the intestinal microbiota during HSCT and a need for targeted microbiota modulation in these patients.

The present invention relates also to a method of tailoring a probiotic composition based on the differences in Lactobacillus/LAB strains found in the intestines of individuals with non-secretor and secretor blood group phenotypes.

The present invention relates to a probiotic composition which is tailored based on the spectrum of Lactobacillus/LAB that can be found in the intestine of non-secretor individuals. The actual Lactobacillus/LAB strains that are missing or lower in abundance in either secretor or non-secretor individuals can be determined using techniques known in the art, for example, by using Denaturating Gradient Gel Electrophoresis and/or systematic DNA-sequencing of samples taken from secretor and non-secretor individuals. The DGGE method is well described in the art (Vanhoutte et al. FEMS Microb Ecol 2004; 48; 437-446; Matsuki et al. Applied and Environmental Microbiology 2004; 70; 7220-7228; Satokari et al. AEM 2001; 67: 504-513; Mättö et al. FEMS Immunol Med Microbiol. 2005; 43: 213-22) It is possible to isolate the bacterium or strain representing a particular PCR-DGGE genotype. For isolation of specific Lactobacillus or LAB genotypes a faecal sample can be cultured on LAB selective culture medium such as Rogosa or LAMVAB. Bacterial isolates can be subcultured from the culture plates and identified to the species level by 16S rDNA sequencing. An isolate is then run in DGGE with known control samples with a particular DGGE band position.

Steps for tailoring a microbial composition typically comprise:
determination of secretor/non-secretor status of an individual using standard methods described in the art;
determination of spectra of Lactobacillus or LAB typical to the secretor and non-secretor phenotypes by determining the microbes found in the intestinal samples of a sufficient number (at least one) of individuals with secretor and non-secretor phenotypes, based on the analyses using DGGE and sequencing the 16S rRNA gene and isolating the relevant strains;
determination of bacteria or strains or bacterial genotypes significantly enriched in samples from secretor or non-secretor individuals by comparing the spectra of microbes between secretor and non-secretor samples;
optionally preparing a bacterial product containing a high amount of the bacteria and/or strains or bacterial genotypes significantly enriched in the desired secretor/non-secretor samples.

Then microbiota of a recipient can be stabilized and/or modified by administering the secretor/non-secretor tailored bacteria or a product thereof to the recipient.

The culturing can also be performed in a device mimicking the gastrointestinal tract. One such is the TNO TIM-1 model. Typically, faecal slurries acquired by mixing faeces with artificial saliva and sterile water are used as an input for the TIM-1 model. The faecal slurries can be prepared from study groups, for example, from pooled non-secretor and secretor samples. In TNO TIM-1 model, various parameters can be adjusted, e.g. level of gastric secretion, time to addition of bile and pancreatin, etc. In each compartment the physiological concentrations of bile salts, pancreatic enzymes and electrolytes simulates an average physiological passage through the small intestine. The survival of targeted bacteria can be compared between the study groups, in order to look for functional differences between bacterial populations.

In addition, the present invention relates to a probiotic composition which is tailored based on the spectrum of Lactobacillus/LAB that can be found in the intestine of secretor individuals. The actual Lactobacillus/LAB strains that are more abundant in the intestine of secretor individuals, for example, can be determined correspondingly. The probiotic composition of the invention contributes in supporting the maintenance of Lactobacillus/LAB diversity and/or abundance and/or repertoire of an individual. In one embodiment, the invention is related to probiotic composition targeted to elderly individuals for supporting the maintenance of Lactobacillus/LAB diversity and/or abundance.

In one embodiment, the microbial or probiotic composition comprises L. plantarum. In another embodiment, the microbial or probiotic composition comprises L. acidophilus. In a further embodiment, the microbial or probiotic composition comprises L. plantarum. and L. acidophilus.

In one embodiment, the microbial or probiotic composition comprises L. plantarum having DGGE band position 6.30%. In another embodiment, the microbial or probiotic composition comprises L. acidophilus having DGGE band position 26.80%. In a further embodiment, the microbial or probiotic composition comprises L. plantarum having DGGE band position 6.30% and L. acidophilus having DGGE band position 26.80%.

In an embodiment, the microbial or probiotic composition comprises L. plantarum having DNA sequence encoding the 16S rRNA identified as SEQ ID NO:1. In another embodiment, the microbial or probiotic composition comprises L. acidophilus having DNA sequence encoding the 16S rRNA identified as SEQ ID NO:2. In a further embodiment, the microbial or probiotic composition comprises L. plantarum having DNA sequence encoding the 16S rRNA identified as SEQ ID NO:1 and L. acidophilus having DNA sequence encoding the 16S rRNA identified as SEQ ID NO:2. In an even further embodiment L. plantarum having DNA sequence encoding the 16S rRNA identified as SEQ ID NO:1 and L. acidophilus having DNA sequence encoding the 16S rRNA identified as SEQ ID NO:2 are the probiotics of the composition.

In an embodiment, the microbial or probiotic composition comprises L. plantarum having DGGE band position 6.30% and DNA sequence encoding the 16S rRNA identified as SEQ ID NO:1. In another embodiment, the microbial or probiotic composition comprises L. acidophilus having DGGE band position 26.80% and DNA sequence encoding the 16S rRNA identified as SEQ ID NO:2. In a further embodiment, the microbial or probiotic composition comprises L. plantarum having DGGE band position 6.30% and DNA sequence encoding the 16S rRNA identified as SEQ ID NO:1 together with L. acidophilus having DGGE band position 26.80% and DNA sequence encoding the 16S rRNA identified as SEQ ID NO:2.

The probiotic compositions and supplements so designed may have beneficial effects on the health and/or well-being of a human and may be in the form of, for example, food, capsule, or powder. The composition can be formulated into a functional food product or a nutritional supplement as well as a capsule, emulsion, or powder.

A typical probiotic ingredient is freeze-dried powder containing typically 10¹⁹-10¹² viable probiotic bacterial cells per gram. In addition it normally contains freeze drying carriers such as skim milk, short sugars (oligosaccharides such as sucrose or trehalose). Alternatively, the culture preparation can be encapsulated by using e.g. alginate, starch, xanthan as a carrier. A typical probiotic supplement or capsule preparation contains approximately 10⁹-10¹¹ viable probiotic bacterial cells per capsule as a single strain or multi-strain combination. A typical probiotic food product, which can be among others fermented milk product or juice, contains approximately 10⁹-10¹¹ viable probiotic bacterial cells per daily dose. Probiotics are incorporated in the product as a probiotic ingredient (frozen pellets or freeze dried powder) or they are cultured in the product during fermentation.

Lactobacillus/LAB enriched composition or supplement contains optionally also at least one prebiotic agent optimised for the growth stimulation of Lactobacillus/LAB strains.

The invention will be described in more detail by means of the following examples. The examples are not to be construed to limit the claims in any manner whatsoever.

### Example 1

58 healthy adult volunteers were recruited to the study. Both faecal and blood samples were collected. The age of the volunteers ranged from 31 to 61 and was in average 45 years.

Secretor status was determined from blood samples by using the standard in-house blood grouping protocols of Finnish Red Cross Blood Service, Helsinki Finland. 49 samples were found to be secretors and six were non-secretors. For 3 samples, secretor status could not be accurately determined serologically from the blood sample.

Faecal samples were frozen within 5 hours from defecation. DNA from 0.3 g of faecal material was extracted by using the FASTDNA® SPIN KIT FOR SOIL (Qbiogene).

PCR-DGGE method was optimised for Lactobacillus-group. Partial eubacterial 16S rRNA gene was amplified by PCR with the group specific primers shown in Table 1. Amplified PCR fragments were separated in 8% DGGE gel with denaturing gradient ranging from 45% to 60%. DGGE gels were run at 70 V for 960 mins. DGGE gels were stained with SYRBSafe for 30 mins and documented with Safelmager Bluelight table (Invitrogen) and AplhaImager HP (Kodak) imaging system.

Digitalised DGGE gel images were imported to the Bionumerics-program version 5.0 (Applied Maths) for normalisation and band detection. Bands were normalised with marker sample specific for above mentioned bacterial groups were constructed from strains. Band search and bandmatching was performed as implemented in Bionumerics. Bands and bandmatching were manually checked and corrected. Principal component analysis was calculated in Bionumerics. Other statistical analysis were computed with statistical programming language R, version 2.8.1.

The bands were excised from DGGE gels. DNA fragments from bands was eluted by incubating the gel slices in 50 μl sterile H₂O at +4° C. overnight. The correct positions and purity of the bands were checked for each excised bands by amplifying DNA in bands and running the amplified fragments along the original samples in DGGE. Bands, which only produced single bands and were in the correct position in the gels, were sequenced. The sequences were trimmed, and manually checked and aligned by ClustalW. The closest relatives of the sequences were searched using Blast and NCBI nr database. Distance matrix of the aligned sequences was used to compare the similarity of the sequences.

TABLE 1
Primers and their sequences used in this study

| Target group | Primer | sequences | Reference |
|---|---|---|---|
| Lactobacillus | Lac1 | AGCAGTAGGGAATCTTCCA | Walter et al. 2001** |
| Lactobacillus | Lac2GC | GC glamp2*-ATTYCACCGCTACACATG | Walter et al. 2001 |

*GC glamp 2 sequence:
CGCCCGCCGCGCCCCGCGCCCGGCCCGCCGCCCCCGCCCC
**Walter et al. 2001, Appl Environ Microbiol. 67: 2578-2

### Results

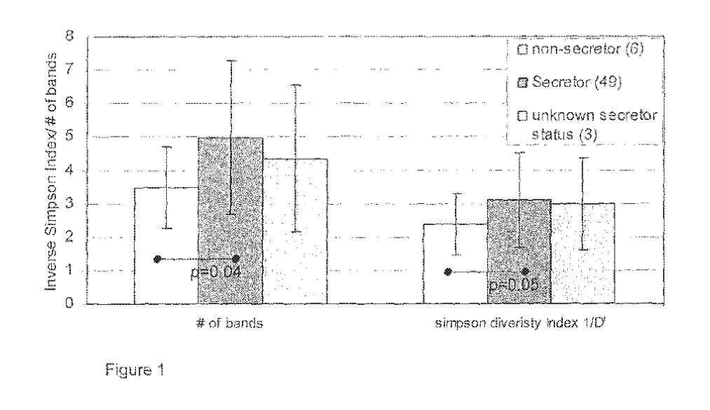

The richness, i.e. the number of bands or genotypes detected and the diversity in Lactobacillus-DGGE differed statistically significantly between the non-secretor and secretor samples. The non-secretor samples had a lower richness than secretor samples (p=0.04). Moreover, the diversity of Lactobacillus was lowered in the non-secretor samples as compared to the secretors (p=0.05; FIG. 1).

### Example 2

In this example, the number of volunteers was increased from 59 volunteers included in example 1 to 71 by recruiting 12 new volunteers. For these 71 volunteers, in addition to phenotyping, secretor status was genotyped by sequencing the coding exon of FUT2 as described in Silva et al. (Glycoconj J 2010, 27, 61-68) and Ferrer-Admetlla et al. (Mol Biol Evol 2009, 26, 1993-2003). Genotyping of FUT2 exon allowed determination of secretor status for the Lewis negative individuals, whose phenotypic secretor status could not be determined. The DGGE analysis and data-analysis were performed as described above. To identify the DGGE band positions, lactobacillar strains were isolated by culturing of faecal samples on lactobacilli selective culture media (Rogosa or LAMVAB) followed by subculturing of colonies. Faecal isolates were identified to species level by 16S rDNA sequencing and to strain level by RAPD or Riboprinter analysis (Mättö et al. J Appl Microbiol 2004; 98; 459-470) and stored in the culture collection of Finnish Red Cross Blood Service. The strains were further analysed in parallel with the faecal DNA samples in DGGE to find the band positions they correspond. In addition to the direct culturing of the samples culturing was performed after pre-treatment with the TNO TIM-1 model, which mimics the conditions in the upper GI-tract. For the pre-treatment faecal slurries acquired by mixing faeces with artificial saliva and sterile water were used as input for the TIM-1 model. The faecal slurries were prepared from pooled non-secretor samples (n=11; total 12.1 g faeces) and secretor samples (n=11; total 9.8 g of faeces) were used. In TNO TIM-1 model T1/2 for emptying the gastric content was set to 20 min, pH change from pH 2.0 to 1.7 in 30 min and level of gastric secretion on 20%. The gastric content was passed into the duodenal compartment, where it was neutralized to pH 6.4, and bile and pancreatin were added, followed passage (time 10 minutes) into the jejunum compartment and into the ileum compartment. In each compartment the physiological concentrations of bile salts, pancreatic enzymes and electrolytes simulated in combination with an average physiological passage through the small intestine. The samples were collected from after 120-180, 180-240 and 240-300 mins treatment. Samples were collected from faecal slurries before the TIM-1 treatment (intake samples) and after the treatments. Dilution series of collected samples were plated in duplicate on applied culturing media and incubated for 72 hours at 37° C.

Statistical analyses, Anova and Fisher's exact test, were computed with statistical programming language R, version 2.10.1.

In the enlarged dataset (n=71), 57 individuals represented secretors and 14 represented non-secretors.

The analysis of the enlarged dataset with DGGE revealed that the incidence and/or band intensity of two Lactobacillus genotypes (i.e. DGGE band positions) were significantly different between groups (see table 2). Both genotypes were more commonly detected in non-secretor individuals than secretor individuals. This indicates that these genotypes are associated to the secretor status of the individual. Lactobacilli strains were recovered from both sample pools by direct culturing and by culturing after pre-treatment of the samples in the TIM-1 model. Based on the cultivation on the LAMVAB, which is a highly specific culture medium for lactobacilli, the survival rate of lactobacilli during the TIM-1 model transit was somewhat higher in the secretor pool than in non-secretor pool (0.6% vs. 0.03%) suggesting higher proportion of acid and bile tolerant lactobacilli in secretors (see table 4).

By analysing the faecal samples in parallel to the identified Lactobacillus strains and comparing the DGGE band positions, the position 26.80% was identified as Lactobacillus acidophilus and position 6.30% as Lactobacillus plantarum.

TABLE 2
The significantly differing band positions of Lactobacillus group
and the incidence of bands in non-secretor (NSS, n = 14) and secretor
samples (SS, n = 57) by PCR-DGGE. P-values for ANOVA
and Fisher's exact test are indicated

| | ANOVA/ | in NSS, | | # of | |
|---|---|---|---|---|---|
| Band position | FISHER | % | in SS, % | hits | Identification |
| 6.30% | 0.001/0.02 | 50% | 12% | 14 | L. plantarum |
| 26.80% | 0.004/0.01 | 57% | 12% | 15 | L. acidophilus |

TABLE 3
Identification by DNA sequencing the 16S rRNA gene of lactobacilli
representing the band positions 6.30% and 26.80% (sequence 5′->3′).

| Band | |
|---|---|
| position | 16S rRNA gene fragment defining the DGGE band position |
| 6.30% | CAATGGACGAAAGTCTGATGGAGCAACGCCGCGTGAGTGAAGAAGGGTTT |
| | CGGCTCGTAAAACTCTGTTGTTAAAGAAGAACATATCTGAGAGTAACTGTT |
| | CAGGTATTGACGGTATTTAACCAGAAAGCCACGGCTAACTACGTGCCAGCA |
| | GCCGCGGTAATACGTAGGTGGCAAGCGTTGTCCGGATTTATTGGGCGTAA |
| | AGCGAGCGCAGGCGGTTTTTTAAGTCTGATGTGAAAGCCTTCGGCTCAAC |
| | CGAAGAAGTGCATCGGAAACTGGGAAACTTGAGTGCAGAAGAGGACAGTG |
| | GAACTC (SEQ ID NO: 1) |
| 26.80% | CAATGGACGAAAGTCTGATGGAGCAACGCCGCGTGAGTGAAGAAGGTTTT |
| | CGGATCGTAAAGCTCTGTTGTTGGTGAAGAAGGATAGAGGTAGTAACTGG |
| | CCTTTATTTGACGGTAATCAACCAGAAAGTCACGGCTAACTACGTGCCAGC |
| | AGCCGCGGTAATACGTAGGTGGCAAGCGTTGTCCGGATTTATTGGGCGTA |
| | AAGCGAGCGCAGGCGGAAGAATAAGTCTGATGTGAAAGCCCTCGGCTTAA |
| | CCGAGGAACTGCATCGGAAACTGTTTTTCTTGAGTGCAGAAGAGGAGAGT |
| | GGAACTC (SEQ ID NO: 2) |

TABLE 4
The survival of lactobacilli from pooled faecal samples of
secretor and non-secretor individuals in the TIM-1 model
(upper gastrointestinal tract conditions). Viability was
determined by plate count culturing using RCBA and BBE media.

| | Secretor pool | | Non-secretor pool | |
|---|---|---|---|---|
| | Rogosa | LAMVAB | Rogosa | LAMVAB |
| Intake, total cfu in | 8.4E+09 | 1.3E+07 | 3.0E+9 | 4.1E+07 |
| sample | | | | |
| Total survival, total cfu | 1.0E+08 | 7.5E+04 | 4.8E+07 | 1.4E+04 |
| in samples | | | | |
| % survival | 1.2 | 0.6 | 1.6 | 0.03 |

### Example 3

The Human Intestinal Tract (HIT) chip (Rajilic-Stojanovic et al. 2009, Environ Microbiol 11 (7): 1736-1751) was applied to more detailed analysis of the microbiota in non-secretor and secretor individuals. HITchip contains approximately 5000 nucleotide probes targeting over 1000 phylotypes of bacteria colonising the human gut. HITChip analysis were performed as described in Rajilic-Stojanovic et al. 2009 for DNA (extracted from 1 g of DNA by the Apajalahti et al. 1998 method) samples of 12 non-secretor and 12 secretor individuals. The data was normalised and analysed in R using within-array spatial normalization and quality control as described in Rajilic-Stojanovic et al. 2009. On top of that between-array normalization was performed with quantile normalization. The differences for each bacterial group between the sample groups were studied with linear models and ANOVA-tests, transforming the array intensities into logarithmic scale first.

In addition, relative abundance of taxa in level 1 (species-like level) were significantly different between non-secretor and secretor individuals based on the ANOVA of hybridisation signals of probes. All of the significantly differing Lactobacillus taxa were relatively more abundant in non-secretor than in secretor samples (Table 5), which is consistent with the DGGE results.

TABLE 5
The Lactobacillus and Weissella group in level 1 (species-like
level), whose relative abundances were significantly differing between
non-secretor and secretor individuals. Only bacterial groups, which had
p-value >0.05 in ANOVA, are shown.

| | | Hybridisation | |
|---|---|---|---|
| Level 1 (=genus-like level) | Level 3 | signal | p-value |
| Lactobacillus jensenii | Bacilli | NSS > SS | 0.02* |
| Lactobacillus sp. KC38 | Bacilli | NSS > SS | 0.01* |
| Lactobacillus antri | Bacilli | NSS > SS | 0.01* |
| Lactobacillus brevis | Bacilli | NSS > SS | 0.02* |
| Lactobacillus mucosae | Bacilli | NSS > SS | 0.05* |
| Lactobacillus oris | Bacilli | NSS > SS | 0.008** |
| Lactobacillus paracasei | Bacilli | NSS > SS | 0.02* |
| Lactobacillus plantarum | Bacilli | NSS > SS | 0.01* |
| uncultured Pediococcus sp. | Bacilli | NSS > SS | 0.04* |
| NS1A12 | | | |
| Weissella cibaria | Bacilli | NSS > SS | 0.02* |
| Weissella confusa | Bacilli | NSS > SS | 0.02* |

1-20. (canceled)

21. A method of tailoring a microbial composition, wherein the composition is tailored based on the spectrum of Lactobacillus/LAB composition found from the intestine of at least one individual with non-secretor/secretor blood group phenotype.

22. The method according to claim 21, wherein the individual is secretor.

23. The method according to claim 21, wherein the individual is non-secretor.

24. A microbial composition comprising L. plantarum having DNA sequence encoding the 16S rRNA identified as SEQ ID NO:1 and/or L. acidophilus having DNA sequence encoding the 16S rRNA identified as SEQ ID NO:2.

25. The microbial composition according to claim 24, wherein the composition comprises L. plantarum having DNA sequence encoding the 16S rRNA identified as SEQ ID NO:1 and L. acidophilus having DNA sequence encoding the 16S rRNA identified as SEQ ID NO:2.

26. The microbial composition according to claim 24 for treating and/or preventing inflammatory bowel disease, diarrhoea, respiratory tract infections, irritable bowel syndrome, atopy/allergy, celiac disease and/or disturbed balance of mucosal microbiota followed by stem cell transplantation and/or subsequent graft-versus-host disease.

27. A method of assessing the need of an individual for Lactobacillus/LAB supplementation or estimating a dose of Lactobacillus/LAB supplementation needed for a desired effect in an individual by determining the secretor/non secretor blood group status of the individual.

28. The method according to claim 27 wherein Lactobacillus/LAB supplementation comprises L. plantarum and/or L. acidophilus.

29. The method according to claim 28 wherein the said supplementation comprises L. plantarum having DNA sequence encoding the 16S rRNA identified as SEQ ID NO:1 and/or L. acidophilus having DNA sequence encoding the 16S rRNA identified as SEQ ID NO:2.

30. A method of augmenting stabilisation of a mucosal Lactobacillus/LAB composition of an individual in disorders related to, or after treatments leading to unbalance of mucosal microbiota by determining the secretor/non secretor blood group status of the individual.

31. The method according to claim 30 wherein the disorder is celiac disease and/or graft-versus-host disease.

32. A method of assessing the need of an individual for supplementation of Lactobacillus/LAB-supporting prebiotic or of estimating a dose of supplementation of Lactobacillus/LAB supporting prebiotic needed for a desired effect by determining the secretor/non secretor blood group status of the individual.


SEQUENCE LISTING (5 sequences)

SEQ ID NO: 1 - DNA, 308 bp, Lactobacillus plantarum
caatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactc

SEQ ID NO: 2 - DNA, 308 bp, Lactobacillus acidophilus
caatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactc

SEQ ID NO: 3 - DNA, 19 bp, Artificial Sequence
  Primer
agcagtagggaatcttcca

SEQ ID NO: 4 - DNA, 18 bp, Artificial Sequence
  Primer
attycaccgctacacatg

SEQ ID NO: 5 - DNA, 40 bp, Artificial Sequence
  Primer
cgcccgccgcgccccgcgcccggcccgccgcccccgcccc